(19) United States
(12) Patent Application Publication
(10) Pub. No.: US 2005/0036954 A1
(43) Pub. Date: Feb. 17, 2005

(54) Combination of toothpaste, a chemical agent and natural herbs for improving sexual performance

(76) Inventor: Arthur Zuckerman, New York, NY (US)

Correspondence Address: EZRA SUTTON, PA, PLAZA 9, 900 ROUTE 9, WOODBRIDGE, NJ, 07095, US

(21) Appl. No.: 10/639,905

(22) Filed: Aug. 12, 2003

(51) Int. Cl. A61K 7/16

(52) U.S. Cl. 424/49; 514/252.16

(57) ABSTRACT

An oral dentifrice composition for improving sexual activity in a human being. The oral dentifrice composition includes an oral dentifrice formulation being selected from the group consisting of toothpaste, tooth powder, mouth and gum powder, dental cream, prophylaxis paste, mouthwash, mouth rinse, mouth spray, lipid or non-lipid oral gel, lozenge, chewing gum, dental tablet, foaming dental tablet, pastille and other oral hygiene materials; a sexual enhancement compound in a dosage amount of 5 mg to 100 mg for enhancing sexual activity of the user; and one or more natural medicinal herbs and botanicals admixed into the oral dentifrice composition for enhancing sexual activity of the user. The natural medicinal herbs and botanicals are selected from the group consisting of catuaba, damiana, epimedium, maca and muria puama.

### FIELD OF THE INVENTION

The present invention relates to a combined composition of toothpaste, a sexual enhancement chemical compound and medicinal herbs for improving sexual activity in males, in addition to enhancing oral hygiene.

### BACKGROUND OF THE INVENTION

Compositions of various types for improving sexual potency in the form of prescription pharmaceuticals, medicinal herbs, and testosterone enhancers are well known in the United States. There remains a need to improve sexual activity in a simple, convenient and socially acceptable manner. One way of doing so is to use toothpaste as the carrier of sexual enhancement chemical compounds and/or herbs into the toothpaste so that when the user brushes his or her teeth, it accomplishes the sexual enhancement and performance of the user. The use of such oral dentifrice compositions having sexual enhancement compounds therein will overcome emotional stress, embarrassment or social stigma associated with the administration of a medicinal oral tablet to aid in the correction of sexual dysfunction.

### DESCRIPTION OF THE PRIOR ART

Sexual enhancement compositions having sildenafil citrate as a primary constituent/ingredient have been generally disclosed in the prior art. For example, PCT Application No. 0067708 issued to Ali discloses the use of oral dentifrice compositions preferably formulated as toothpaste or mouthwash containing sildenafil citrate in combination with or without testosterone.

PCT Application No. 03015757 issued to Gruneich discloses the use of formulations suitable for oral administration such as toothpaste, mouthwash, or gels containing sildenafil citrate as one of approximately thirty (30) possible active chemical agents.

None of the prior art references teach or disclose the composition of the present invention of a sexual enhancement oral dentifrice composition having an oral dentifrice formulation such as a toothpaste or mouthwash, a sexual enhancement compound, a sexual enhancement medicinal herb or botanical admixed with the sexual enhancement compound.

Accordingly, it is an object of the present invention to provide a combined composition of an oral dentifrice material (such as toothpaste, mouthwash and the like), a sexual enhancement drug (such as sildenafil citrate) being admixed with medicinal herbs or botanicals for improving sexual activity in males and females.

Another object of the present invention is to provide a sexual enhancement composition that allows the use of such oral dentifrice compositions having sexual activity enhancers therein for overcoming emotional stress, embarrassment or social stigma with the administration of a medicinal oral tablet to aid in correction of sexual dysfunction.

Another object of the present invention is to provide a sexual enhancement composition that uses an oral dentifrice composition having a therapeutically effective amount of sexual activity enhancers therein, wherein the oral dentifrice composition include compositions formulated as a toothpaste, tooth powder, mouth and gum powder, dental cream, prophylaxis paste, mouthwash, mouth rinse, mouth spray, lipid or non-lipid oral gel, lozenge, chewing gum, dental tablet, foaming dental tablet or pastille.

Another object of the present invention is to provide a sexual enhancement composition that uses an oral dentifrice composition having a therapeutically effective amount of sexual activity enhancers therein, wherein the sexual activity enhancers further include medicinal herbs or botanicals.

Another object of the present invention is to provide a sexual enhancement composition that uses an oral dentifrice composition having a therapeutically effective amount of sexual activity enhancers therein, wherein the sexual activity enhancers include sildenafil citrate, vardenafil hydrochloride or tadalafil, or sildenafil citrate, vardenafil hydrochloride or tadalafil admixed with medicinal herbs or botanicals which produces smooth muscle relaxation of the corpus cavemosurn and allows inflow of blood to the penis, followed by an erection of the penis for increased sexual pleasure.

A further object of the present invention is to provide a sexual enhancement composition that has a minimal amount of side effects, is fast acting and easy to use.

A still further object of the present invention is to provide a sexual enhancement composition that can be mass produced in an automated, hygienic and economical manner and is readily affordable by the user.

### SUMMARY OF THE INVENTION

Sexual arousal triggers a surge in cyclic GMP, a natural penile blood flow activator that governs the vigor of a man's erection during intercourse. After orgasms under the influence of an enzyme called phosphoediesterase-5 (PDE-5), cyclic GMP levels fall and a man's erection fades. Sildenafil citrate, vardenafil hydrochloride and tadalafil are all PDE-5 inhibitors and work to bolster cyclic GMP levels so that a man can have a firmer more rigid erection during sex.

In accordance with the present invention, there is provided an oral dentifrice composition for improving sexual activity in a human being. The oral dentifrice composition includes an oral dentifrice formulation being selected from the group consisting of toothpaste, tooth powder, mouth and gum powder, dental cream, prophylaxis paste, mouthwash, mouth rinse, mouth spray, lipid or non-lipid oral gel, lozenge, chewing gum, dental tablet, foaming dental tablet, pastille and other oral hygiene materials; a sexual enhancement compound in a dosage amount in the range of 5 mg to 200 mg for enhancing sexual activity of the user; and one or more natural medicinal herbs and botanicals admixed into the oral dentifrice composition for enhancing sexual activity of the user. The sexual enhancement compound is selected from the group consisting of sildenafil citrate, vardenafil hydrochloride, and tadalafil. The natural medicinal herbs and botanicals are selected from the group consisting of catuaba, damiana, horny goat weed, maca and muria puama.

### DETAILED DESCRIPTION OF THE PREFERRED AND ALTERNATE EMBODIMENTS

### Overview

The oral dentifrice composition for enhancing and improving sexual activity includes an oral dentifrice formulation, a sexual enhancement pharmalogical compound in the form of sildenafil citrate, vardenafil hydrochloride or tadalafil for enhancing sexual activity of a user, and one or more natural medicinal herbs and botanicals admixed into the oral dentifrice composition for also enhancing sexual activity of the user.

As used herein, the term “oral dentifrice composition” will be understood to include formulation as a toothpaste, tooth powder, mouth and gum powder, dental cream, prophylaxis paste, mouthwash, mouthrinse, mouth spray, lipid or non-lipid oral gel, lozenge, chewing gum, dental tablet, foaming dental tablet or pastille. Dental floss and toothpicks are not included in the term “oral dentifrice composition” as used herein. In preferred aspects of the present invention, the oral dentifrice composition is formulated as a toothpaste or mouthwash.

Sildenafil citrate, also known as Viagra™, from Pfizer, Inc. of New York, N.Y. is used in providing a therapeutically active ingredient that is effective in activating the enzyme guanylate cyclase, increasing serum levels of cyclic guanosine monophosphate (cGMP), which produces smooth muscle relaxation of the corpus cavernosurn and allows inflow of blood, followed by erection of the penis. Sildenafil citrate is a white crystalline powder, and is soluble in water. Sildenafil citrate is chemically described as 1-[[3-(6,7-dihy-dro-I -methyl-7-oxo-3-propyl-IH-pyrazolo[4,3-d]pyrimidin-5-yi)-4-ethoxyphenyl]sulfonyl]-4-methylpiperazine citrate, having a molecular weight of 666.7. Vardenafil hydrochloride, also known as LEVITRA™ by BAYER or NUVIVA™ by GSK, can be used as a sexual enhancement composition in place of the sildenafil citrate. Tadalafil, also known as CIALIS™ by ELI LILLY, can also be used as a sexual enhancement composition in place of the sildenafil citrate.

An average dosage of toothpaste on a users toothbrush is defined herein as 2 grams or 2000 mg for an average dosage. The dosage amount of the sildenafil citrate is in the range of 12 mg to 100 mg with a preferred dosage amount of 50 mg (2.5% by weight of the average user toothpaste dosage amount). The dosage amount of the vardenafil hydrochloride is in the range of 5 mg to 40 mg with a preferred dosage amount of 20 mg (1.0% by weight of the average user toothpaste dosage amount). The dosage amount of the tadalafil is in the range 5 mg to 25 mg with a preferred dosage amount of 20 mg (1.0% by weight of the average user toothpaste dosage amount).

Further it will be understood that use of the oral dentifrice composition, preferably formulated as a toothpaste, comprising sildenafil citrate, vardenafil hydrochloride or tadalafil admixed with the natural medicinal herbs or botanicals by the user should occur about 2 hours to about one-half hour before sexual activity due to rapid absorption of the sexual enhancement compound.

It is contemplated that the use of such compositions will overcome any emotional distress, embarrassment or social stigma associated with the administration of a medicinal oral tablet to aid in the correction of sexual dysfunction as well as overcome any possible side effects experienced by subjects who orally ingest the tablet form of sildenafil citrate.

It is understood that the sildenafil citrate, vardenafil hydrochloride or tadalafil and the natural medicinal herbs or botanicals will be absorbed through the mucous membranes of the mouth when using the toothpaste or other formulations.

As used herein, the term “natural medicinal herbs or botanicals” for sexual enhancement will be understood to include catuaba, damiana, horny goat weed, maca and muria puama.

### Toothpaste Formulation

The toothpaste composition has base ingredients which comprise a combination of known amounts of: vegetable glycerin; sorbitol; hydrated silica; purified water; xylitol; carrageenan; sodium lauryl sulfate; titanium dioxide; propylparaben; methylparaben; sodium benzoate; and a flavoring agent.

### Embodiment 1

### Toothpaste having Sexual Enhancement Compounds

| | |
|---|---|
| INGREDIENTS | PERCENT % |

A. BASIC TOOTHPASTE BASE

| | |
|---|---|
| Vegetable glycerin | 10.00-30.00 |
| Sorbitol | 10.00-30.00 |
| Hydrated silicia | 10.00-30.00 |
| Purified water | 10.00-30.00 |
| Xylitol | 10.00-30.00 |
| Carrageenan | 1.00-3.00 |
| Sodium lauryl sulfate | 1.00-3.00 |
| Titanium dioxide | 1.00-3.00 |
| Oil of wintergreen | 0.300-1.00 |

B. SEXUAL ENHANCEMENT COMPOUNDS

| INGREDIENT | Range | Preferred amount | Percent |
|---|---|---|---|
| Sildenafil | 12 mg to 100 mg per | 50 mg per unit dosage | 2.5% |
| citrate | unit dosage | | |
| Vardenafil | 5 mg to 40 mg per | 20 mg per unit dosage | 1.0% |
| hydrochloride | unit dosage | | |
| Tadalafil | 5 mg to 25 mg per | 20 mg per unit dosage | 1.0% |
| | unit dosage | | |

C. MEDICINAL HERB OR BOTANICAL

| | | | |
|---|---|---|---|
| Catuaba | 20 mg to 100 mg | 60 mg per unit dosage | 3.0% |
| (Juniperis | per unit dosage | | |
| brasilieneis) | | | |
| Damiana | 20 mg to 100 mg | 60 mg per unit dosage | 3.0% |
| (Turnera | per unit dosage | | |
| aphrosisraca) | | | |
| Horny Goat | 20 mg to 100 mg | 60 mg per unit dosage | 3.0% |
| Weed | per unit dosage | | |
| (Epimedium) | | | |
| Maca | 20 mg to 100 mg | 60 mg per unit dosage | 3.0% |
| (Lipidium | per unit dosage | | |
| meyenii) | | | |
| Muria Puama | 20 mg to 100 mg | 60 mg per unit dosage | 3.0% |
| | per unit dosage | | |

It is understood that only one (1) of the three (3) sexual enhancement compounds are used within a given toothpaste formulation. All five (5) medicinal herbs are used within a given toothpaste formulation for a sexual enhancement synergism of the medicinal herb ingredients used in the above composition.

### Advantages of the Present Invention

Accordingly, an advantage of the present invention is that it provides for a combined composition of an oral dentifrice material (such as toothpaste, mouthwash and the like), a sexual enhancement drug (such as sildenafil citrate) being admixed with medicinal herbs or botanicals for improving sexual activity in males and females.

Another advantage of the present invention is that it provides for a sexual enhancement composition that allows the use of such oral dentifrice compositions having sexual activity enhancers therein for overcoming emotional stress, embarrassment or social stigma associated with the administration of a medicinal oral tablet to aid in the correction of sexual dysfunction.

Another advantage of the present invention is that it provides for a sexual enhancement composition that uses an oral dentifrice composition having a therapeutically effective amount of sexual activity enhancers therein, wherein the oral dentifrice composition include compositions formulated as a toothpaste, tooth powder, mouth and gum powder, dental cream, prophylaxis paste, mouthwash, mouth rinse, mouth spray, lipid or non-lipid oral gel, lozenge, chewing gum, dental tablet, foaming dental tablet or pastille.

Another advantage of the present invention is that it provides for a sexual composition that uses an oral dentifrice composition having a therapeutically effective amount of sexual activity enhancers therein, wherein the sexual activity enhancers further include medicinal herbs or botanicals.

Another advantage of the present invention is that it provides for a sexual enhancement composition that uses an oral dentifrice composition having a therapeutically effective amount of sexual activity enhancers therein, wherein the sexual activity enhancers include sildenafil citrate, vardenafil hydrochloride or tadalafil admixed with medicinal herbs or botanicals which produces smooth muscle relaxation of the corpus cavernosurn and allows inflow of blood to the penis, followed by an erection of the penis for increased sexual pleasure.

A further advantage of the present invention is that it provides for a sexual enhancement composition that has a minimal amount of side effects, is fast acting and easy to use.

A still further advantage of the present invention is that it provides for a sexual enhancement composition that can be mass produced in an automated, hygienic and economical manner and is readily affordable by the user.

A latitude of modification, change, and substitution is intended in the foregoing disclosure, and in some instances, some features of the invention will be employed without a corresponding use of other features. Accordingly, it is appropriate that the appended claims be construed broadly and in a manner consistent with the spirit and scope of the invention herein.

1. An oral dentifrice composition for improving sexual activity in a human being, comprising:
a) an oral dentifrice formulation being selected from the group consisting of toothpaste, tooth powder, mouth and gum powder, dental cream, prophylaxis paste, mouthwash, mouth rinse, mouth spray, lipid or non-lipid oral gel, lozenge, chewing gum, dental tablet, foaming dental tablet, pastille and other oral hygiene materials;
b) a sexual enhancement compound in a dosage amount in the range of 5 mg to 100 mg for enhancing sexual activity of the user; and
c) one or more natural medicinal herbs and botanicals admixed into said oral dentifrice composition for enhancing sexual activity of the user.

2. An oral dentifrice composition in accordance with claim 1, wherein said sexual enhancement compound is selected from the group consisting of sildenafil citrate, vardenafil hydrochloride, and tadalafil.

3. An oral dentifrice composition in accordance with claim 2, wherein said sildenafil citrate is in a dosage amount in the range of 12 mg to 100 mg.

4. An oral dentifrice composition in accordance with claim 2, wherein said sildenafil citrate is in a preferred dosage amount of 50 mg.

5. An oral dentifrice composition in accordance with claim 2, wherein said vardenafil hydrochloride is in a dosage amount in the range of 5 mg to 40 mg.

6. An oral dentifrice composition in accordance with claim 2, wherein said vardenafil hydrochloride is in a preferred dosage amount of 20 mg.

7. An oral dentifrice composition in accordance with claim 2, wherein said tadalafil is in a dosage amount in the range of 5 mg to 25 mg.

8. An oral dentifrice composition in accordance with claim 2, wherein said tadalafil is in a preferred dosage amount of 20 mg.

9. An oral dentifrice composition in accordance with claim 1, wherein said natural medicinal herbs and botanicals are selected from the group consisting of catuaba, damiana, epimedium, maca and muria puam.

10. An oral dentifrice composition in accordance with claim 1, wherein said natural medicinal herbs are in a dosage amount in the range of 20 mg to 100 mg.

11. An oral dentifrice composition in accordance with claim 1, wherein said natural medicinal herbs are in a preferred dosage amount of 60 mg.

12. An oral dentifrice composition for improving sexual activity in a human being, comprising:
a) an oral dentifrice formulation being selected form the group consisting of toothpaste, tooth powder, mouth and gum powder, dental cream, prophylaxis paste, mouthwash, mouth rinse, mouth spray, lipid or non-lipid oral gel, lozenge, chewing gum, dental tablet, foaming dental tablet, pastille and other oral hygiene materials; and
b) one or more natural medicinal herbs and botanicals selected from the group consisting of catuaba, damiana, epimedium, maca and muria puam.
